(19) United States
(12) Patent Application Publication
(10) Pub. No.: US 2025/0109368 A1
(43) Pub. Date: Apr. 3, 2025

(54) SYSTEM AND PROCESS FOR DECELLULARIZING BLOOD VESSELS

(71) Applicant: LifeNet Health, Virginia Beach, VA (US)

(72) Inventors: Adam Entsminger, Virginia Beach, VA (US); Dominic Squillace, Virginia Beach, VA (US); Jason Schulte, Virginia Beach, VA (US)

(21) Appl. No.: 18/728,343

(22) PCT Filed: Jan. 11, 2023
(86) PCT No.: PCT/US2023/010553

§ 371 Date: Jul. 11, 2024

Related U.S. Application Data
(60) Provisional application No. 63/298,780, filed on Jan. 12, 2022.

(51) Int. Cl. C12M 1/12; C12M 1/00; C12N 5/071

(52) U.S. Cl. CPC C12M 23/06; C12M 23/34; C12M 29/20; C12N 5/0691

(57) ABSTRACT

A system and method for processing a tubular tissue sample defining a lumen and an exterior surface. The system includes a container having a one or more walls defining a hollow interior chamber for receiving the tubular tissue sample. A first fluid inlet passage is associated with the container and configured to direct fluid through the lumen of the tissue sample. A second fluid inlet passage is also associated with the container and configured to direct fluid over the exterior surface of the tissue sample.

### CROSS REFERENCE TO RELATED APPLICATIONS

This application claims priority from U.S. Provisional Application Ser. No. 63/298,780, titled “SYSTEM AND PROCESS FOR DECELLULARIZING BLOOD VESSELS,” filed Jan. 12, 2022, incorporated herein by reference in its entirety for all purposes.

### FIELD OF THE INVENTION

The present invention relates to a system for decellularizing blood vessels, as well as a process for decellularization.

### BACKGROUND OF THE INVENTION

As is described in U.S. Pat. No. 10,987,449, which is incorporated herein by reference in its entirety and for all purposes, to be successfully utilized for implantation, natural tissue (e.g., allograft and xenograft) is decellularized to remove the native cells and other potential immunogenic material and leave only the non-immunogenic structural materials (collagen, elastin, laminin, etc.).

Typical decellularization methods include a series of chemical (e.g., detergent or enzymatic) washes that can remove the cells by immersion.

While typical immersion methods can completely decellularize relatively thin and flat tissue samples, it can be more difficult to completely decellularize tubular body structures, such as veins and arteries, due to their tubular geometries. In view of the foregoing, described herein is a system and method for decellularizing tissue having tubular structures.

### SUMMARY OF THE INVENTION

According to one aspect of the invention, a system for processing a tubular tissue sample defining a lumen and an exterior surface. The system includes a container having one or more walls defining a hollow interior chamber for receiving the tubular tissue sample. A first fluid inlet passage (e.g., 52a) is associated with the container and configured to direct fluid through the lumen of the tissue sample. A second fluid inlet passage (e.g., 52c) is also associated with the container and configured to direct fluid over the exterior surface of the tissue sample.

According to another aspect of the invention, a method for processing a tubular tissue sample that is stored in a container having one or more walls defining a hollow interior chamber, said method comprises:
delivering fluid through a first fluid inlet passage (e.g., 52a) associated with the container and through a lumen of the tissue sample; and
delivering fluid through a second fluid inlet passage (e.g., 52c) associated with the container and over an exterior surface of the tissue sample.

According to yet another aspect of the invention, a tubular tissue product is produced by a process comprising:
delivering fluid through a first fluid inlet passage associated with a container and through a lumen of a tissue sample that is positioned within the container; and
delivering fluid through a second fluid inlet passage associated with the container and over an exterior surface of the tissue sample.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 depicts an isometric view of a system (shown assembled) for decellularizing blood vessels (“system”, hereinafter), as viewed from an outlet side of the system.

FIG. 2 depicts an isometric view of the system, as viewed from an inlet side of the system.

FIG. 3 depicts an inlet-side elevation view of the system.

FIG. 4 depicts a top plan view of the system.

FIG. 5 depicts a cross-sectional view of the system of FIG. 4 taken along the lines 5-5.

FIG. 6 depicts a side elevation view of the system.

FIGS. 7-9 depict cross-sectional views of the system of FIG. 6 taken along the lines 7-7, 8-8 and 9-9, respectively.

FIG. 10 depicts an outlet-side isometric view of the system, which the tube removed to reveal the internal features of the system.

FIG. 11 depicts an exploded view of the system.

FIGS. 12A-12E depict isometric, side elevation, rear elevation, plan, and front elevation views, respectively, of the inlet cap of the system.

FIGS. 12F and 12G depict cross-sectional views of the inlet cap taken along the lines 12F-12F and 12G-12G, respectively.

FIGS. 13A and 13B depict isometric and plan views, respectively, of the outlet cap of the system.

FIG. 13C depicts a cross-sectional view of the outlet cap of FIG. 13B taken along the lines 13C-13C.

FIG. 13D is a detailed view of the outlet cap of FIG. 13C.

FIG. 14 depicts an isometric view of a tube of the system.

FIG. 15 depicts an isometric view of a diffuser of the system.

It is noted that the scaling throughout the figures may not be uniform.

### DETAILED DESCRIPTION OF THE INVENTION

Corresponding reference characters indicate corresponding parts throughout the several views. The exemplification set out herein illustrates an embodiment of the invention, in one form, and such exemplification is not to be construed as limiting the scope of the invention in any manner.

Inasmuch as various components and features of the system described herein are of well-known design, construction, and operation to those skilled in the art, the details of such components and their operations will not generally be discussed in significant detail unless considered of pertinence to the present invention or desirable for purposes of better understanding.

The fluid described herein used for decellularization may be any fluid known to those skilled in the art. As is described in U.S. Pat. No. 7,338,757, which is incorporated by reference herein in its entirety, the fluid may be an anionic agent, for example sodium dodecylsulfate (SDS).

In the drawings, like numerals refer to like items, certain elements and features may be labeled or marked on a representative basis without each like element or feature necessarily being individually shown, labeled, or marked, and certain elements are labeled and marked in only some, but not all, of the drawing figures.

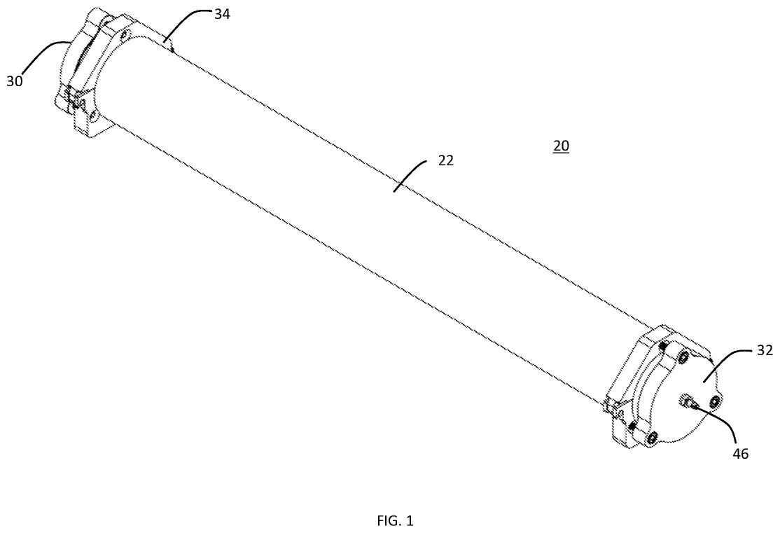

FIGS. 1-11 depict a system for decellularizing blood vessels (“system” hereinafter) according to one exemplary embodiment of the invention. The system is designated by numeral 20, and the blood vessel grafts (two shown) are designated by numeral 21. At the outset it is noted that system 20 is configured to simultaneously decellularize two grafts 21 (as shown), however, it will be understood that system 20 may be modified to decellularize any number of grafts 21. Graft 21 may be an artery, vein, tube, etc. Also, system 20 may be modified (or otherwise used) to decellularize tissue structures other than tubular vessel structures.

As best viewed in FIG. 11, system 20 generally comprises a hollow cylindrical tube 22 having an open inlet end 24 and an open outlet end 26. Tube 22 defines a hollow interior region defining a chamber 28 (FIGS. 7 and 9). It is noted at the outset that tube 22 is not limited to having a circular shape, and therefore, may also be referred to herein more generally as a container.

An inlet cap assembly 30 is mounted over inlet end 24 of tube 22. Similarly, an outlet cap assembly 32 is mounted over outlet end 26 of tube 22. A seal 33 (e.g., O-ring, face seal, piston rod seal, cylinder bore seal, etc.) is mounted between each cap assembly 30, 32 and its respective tube end 24 and 26, respectively, to prevent fluid from inadvertently escaping from tube 22. Each seal 33 may be positioned at least partially within a dove-tailed recess formed in the mounting surface of the respective cap assemblies 30, 32.

Each cap assembly 30, 32 includes a substantially circular plate having three flanges extending from a perimeter thereof. Each flange includes a hole that registers with a hole disposed on a hinge clamp assembly 34. Fasteners 36 are threaded through each set of registered holes for mounting the cap assembly 30, 32 to its respective hinge clamp assembly 34. Each hinge clamp assembly 34 comprises a two-part hinged structure that is clamped to an annular channel 35 formed on a respective end of tube 22. The individual parts of the two part hinged structure are fixed together by a fastener 38. It should be understood that clamp assemblies 34 are fixedly mounted to tube 22 once installed thereon. It should be understood that the invention is not limited to the details of the cap and clamp assemblies, as the mechanical interconnection between those components could vary greatly.

Turning now to the features of the inlet cap assembly 30, the inlet cap assembly 30 generally comprises a cap 50, fittings 44, seals for fittings 44, seal 33, fasteners 36, diffuser 72, seals 73, connectors 86 and 88, and seals for connectors 86.

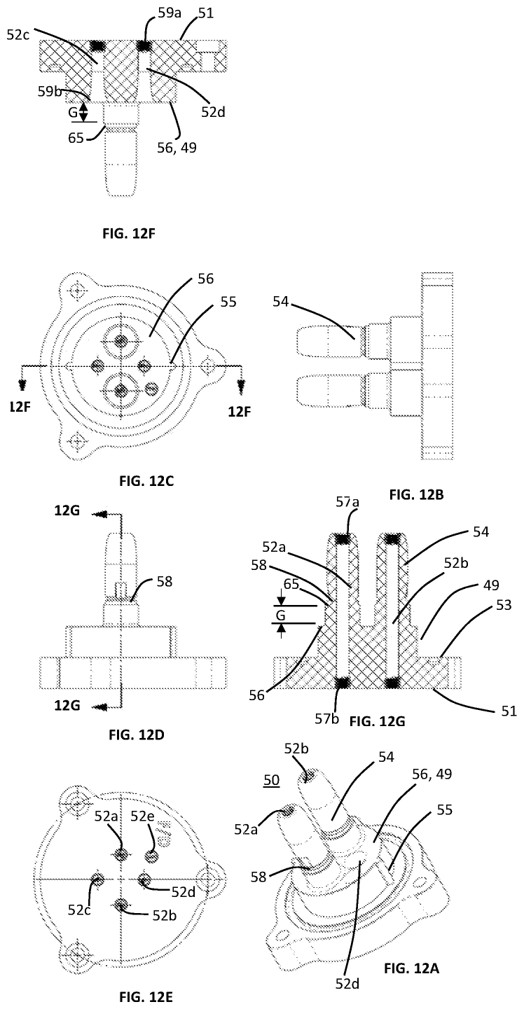

As shown in FIGS. 12A-12G, cap 50 may be a monolithic and unitary body, as shown, or, alternatively, cap 50 may be formed from separate components that are mounted together. Cap 50 has a flat exterior facing surface 51, and a flat interior facing mounting surface 53 (i.e., opposite surface 51) that is configured to bear against inlet end 24 of tube 22. Mounting surface 53 includes a dovetail channel for receiving an O-ring 33, as was previously described. A cylindrical portion 49 extends axially from surface 53. Portion 49 is sized to fit snugly with inlet end 24 of tube 22. An intermediate bearing surface 56 is formed at the free end of portion 49. Bearing surface 56 is spaced apart from surface 53 along axis A. It is noted that surfaces 53 and 56 are oriented parallel to each other. It is also noted that the diameter of surface 56 is less than that of surface 53.

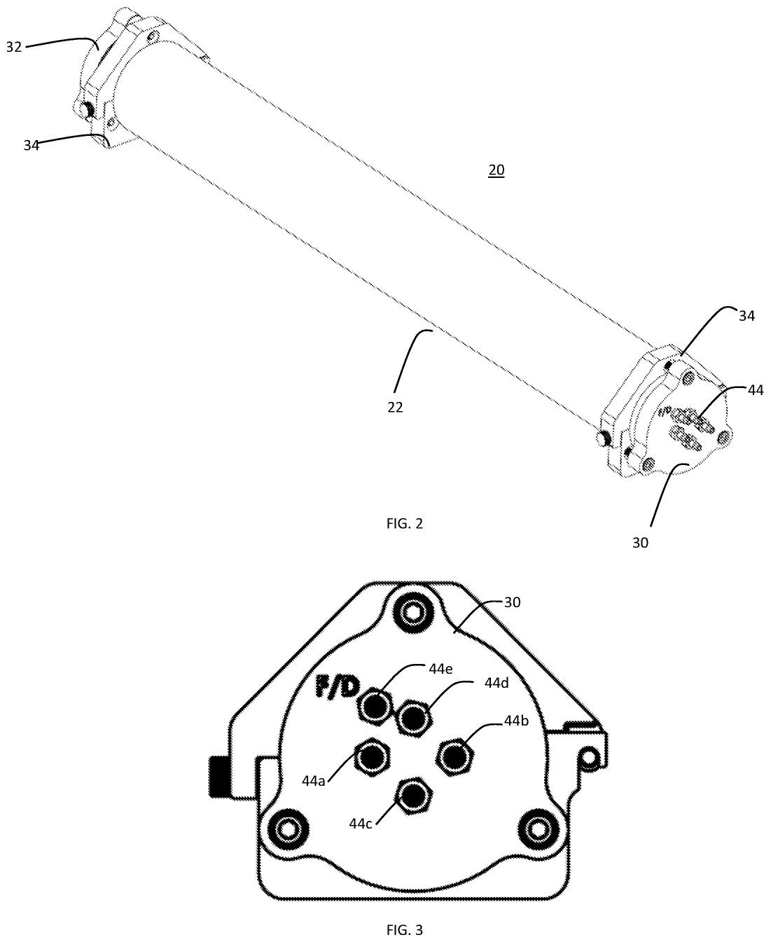

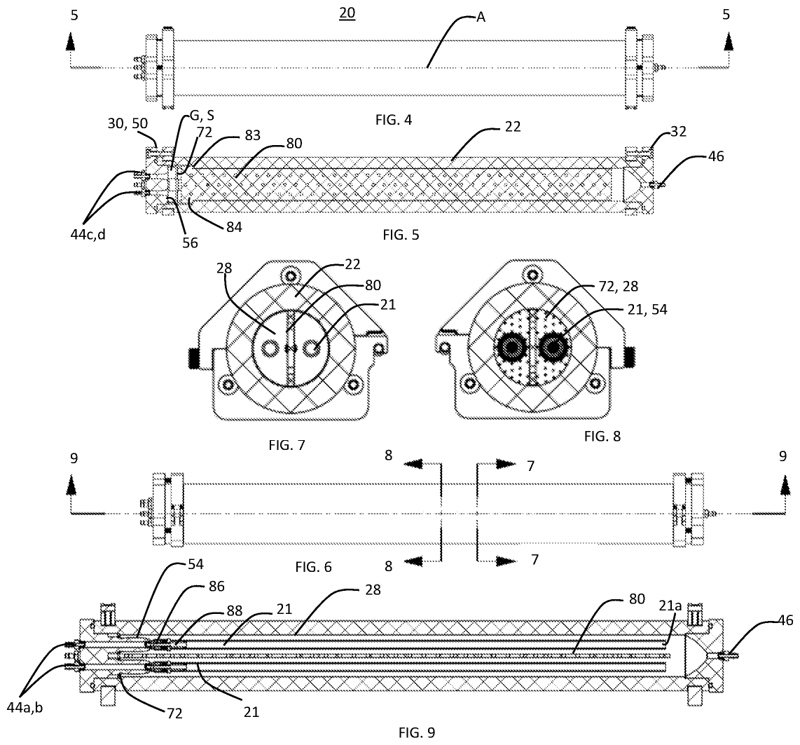

As best shown in FIG. 12G, two hollow and cylindrical protuberances 54 extend from surface 56 in an axial direction (i.e., along longitudinal axis A (FIG. 4) of tube 22 and system 20). The base of each protuberance 54 has an enlarged diameter such that a radially extending shoulder 65 is formed near the base of the protuberance 54. Also, as best shown in FIG. 12G as well as FIG. 5, an axially extending gap ‘G’ is disposed between shoulder 65 and surface 56. The significance of the gap G will be described later. Each protuberance 54 also includes an annular channel 58 formed on the revolved side wall. Channel 58 is positioned close to shoulder 65.

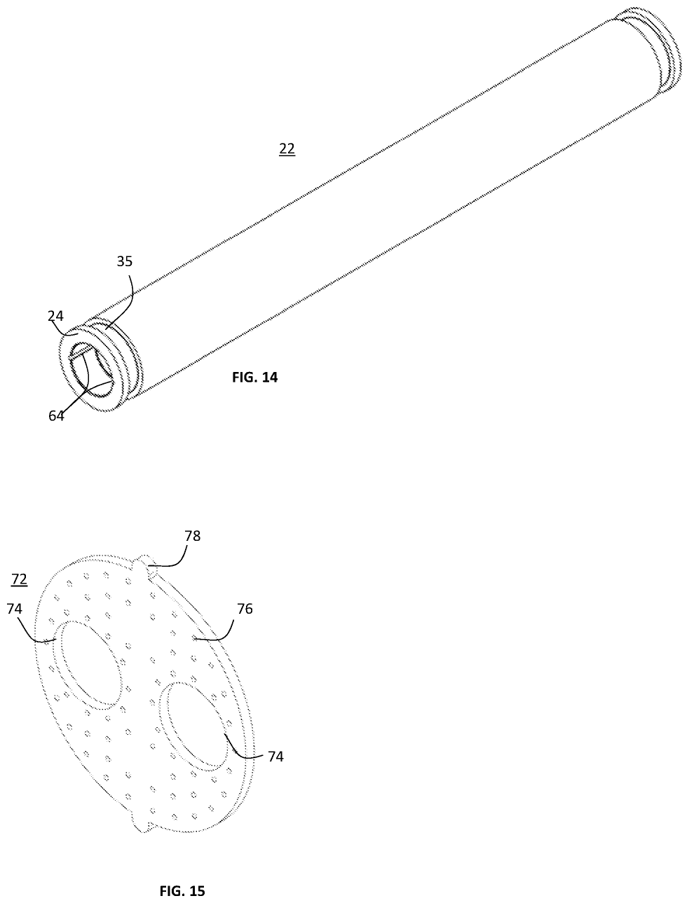

Two diametrically opposed tabs 55 (FIG. 12C) extend outwardly from the perimeter of portion 49. In the assembled state of system 20, tabs 55 are positioned within respective slots 64 (FIG. 14) formed at the inlet end 24 of the interior revolved surface of tube 22 such that cap 50 is non-rotatably aligned with tube 22. Although not shown, the outlet end 26 of tube 22 may also include complimentary slots 64.

Cap 50 includes a series of five fluid passages 52a-52e (referred to either individually or collectively as fluid passage(s) 52) passing therethrough. Each passage 52 is a hole or opening that passes through the entire thickness of cap 50.

As best shown in FIG. 12G, passages 52a and 52b pass through respective protuberances 54. Passages 52a and 52b may have a constant diameter, for example. Each passage 52a and 52b extends between a first end 57a and a second end 57b. First end 57a is also at the free end of a protuberance 54. Second end 57b is disposed on the exterior-facing surface of cap 50 and is configured to be connected to one of the inlet fittings 44 (as will be described later). Ends 57a and 57b may be threaded, for example.

As best shown in FIG. 12F, passages 52c and 52d extend between surfaces 51 and 56. An imaginary line connecting passages 52c and 52d is oriented perpendicular to an imaginary line connecting passages 52a and 52b, such that passages 52a-52d form a diamond pattern, as viewed in FIG. 12E. Each passage 52c and 52d has a first end 59a (otherwise referred to as a fluid entrance) at surface 51 that is threaded and configured to be connected to one of the fittings 44; and a second end 59b (otherwise referred to as an exit port or fluid exit) at surface 56 that is enlarged with respect to first end 59a. The enlarged second end 59b is expanded or enlarged to reduce the pressure of the fluid exiting therefrom. Thus, passages 52c and 52d do not have a constant diameter or cross-section. Alternatively, passages 52c and 52d could have a constant diameter and cross-section. Passage 52e also extends between surfaces 51 and 56.

As best shown in FIG. 3, five separate fluid fittings 44a-44e are mounted to respective passages 52a-52e in inlet cap assembly 30. Fitting 44a is configured to be connected to the end 57b of passage 52a (for delivering fluid through the lumen of one of the grafts 21); fitting 44b is configured to be connected to the end 57b of passage 52b (for delivering fluid through the lumen of the other graft 21); fitting 44c is connected to passage 52c for delivering fluid into chamber 28 of tube 22; fitting 44d is connected to passage 52d for delivering fluid into chamber 28 of tube 22; and fitting 44e is connected to passage 52e for releasing any trapped air bubbles that may develop within chamber 28 of tube 22. It is noted that the location and arrangement of fittings 44 may vary.

Inlet cap assembly 30 includes four connectors 86 and 88 for delivering fluid into the lumens of two grafts 21. More particularly, one hollow-chamber fluid flow connector 86 is connected to end 57a of one of the protuberances 54. An O-ring may be sandwiched between end 57a and connector 86 to prevent the inadvertent escape of fluid at that interface. A second connector 86 is connected to end 57a of the other protuberance 54. Each connector 86 includes a first connection end, which may be threaded, for connection to end 57a of protuberance 54, and a second connection end in the form of a female luer. A hollow chamber for transporting fluid is defined between the first and second connection ends of connector 86.

A hollow-chamber fluid flow connector 88 is connected to the second connection end of one of the connectors 86. Similarly, a hollow-chamber fluid flow connector 88 is connected to the second connection end of the other connector 86. Each connector 88 includes a first connection end, which may include a male luer, for connection to the second connection end of one of the connectors 86, and a second connection end in the form of a barb connector for direct connection to one of the grafts 21. A hollow chamber for transporting fluid is defined between the first and second connection ends of connector 88. Each connector 88 may be a female luer lock to barb connector, for example. It should be understood that the connectors 86 and 88 can vary and are not limited to that which is shown and described. Also, the mated connectors 86 and 88 could be combined into a single connector, if so desired. Furthermore, connectors 86 and 88 could be omitted in their entirety, if so desired, if end 57a of each protuberance were configured to be directly connected to a graft 21.

Turning now to the features of the outlet cap assembly 32 of the system 20, the outlet cap assembly 32 includes a cap 60, seal 33, fasteners 36, fitting 46, and a seal for fitting 46.

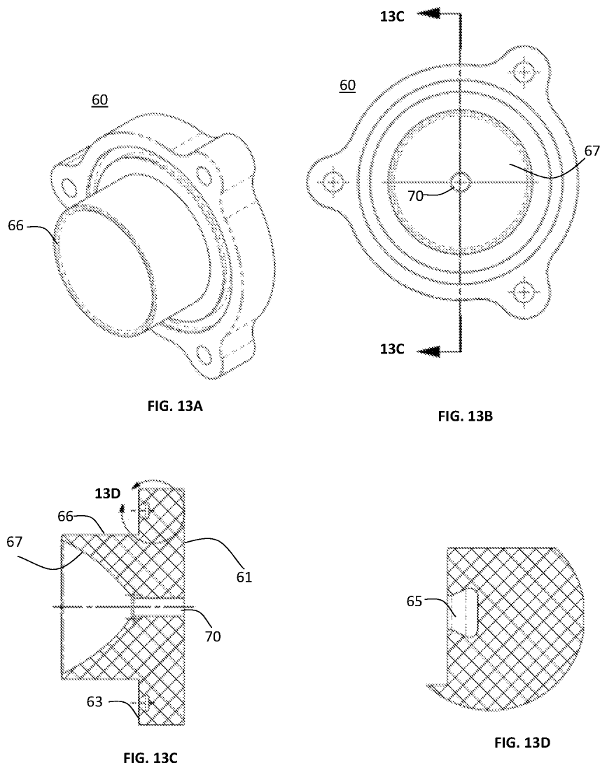

As best shown in FIGS. 13A-13D, cap 60 may be a monolithic and unitary body, as shown, or, alternatively, cap 60 may be formed from separate components that are mounted together. Cap 60 has a flat exterior facing surface 61, and a flat interior facing mounting surface 63 that is configured to bear against outlet end 26 of tube 22. Mounting surface 63 includes a dovetail channel 65 (FIG. 13D) for receiving an O-ring 33, as was previously described. A cylindrical protrusion 66 extends from surface 63, and a concave surface 67 is defined on (or at) the interior facing end of protrusion 66. Protrusion 66 is sized to fit snugly with outlet end 26 of tube 22. Cap 60 includes a single fluid passage 70, in the form of a hole or opening, passing through surface 61 and communicating with concave surface 67. The end of passage 70 at surface 61 may or may not be threaded. Outlet fitting 46 is connected to passage 70.

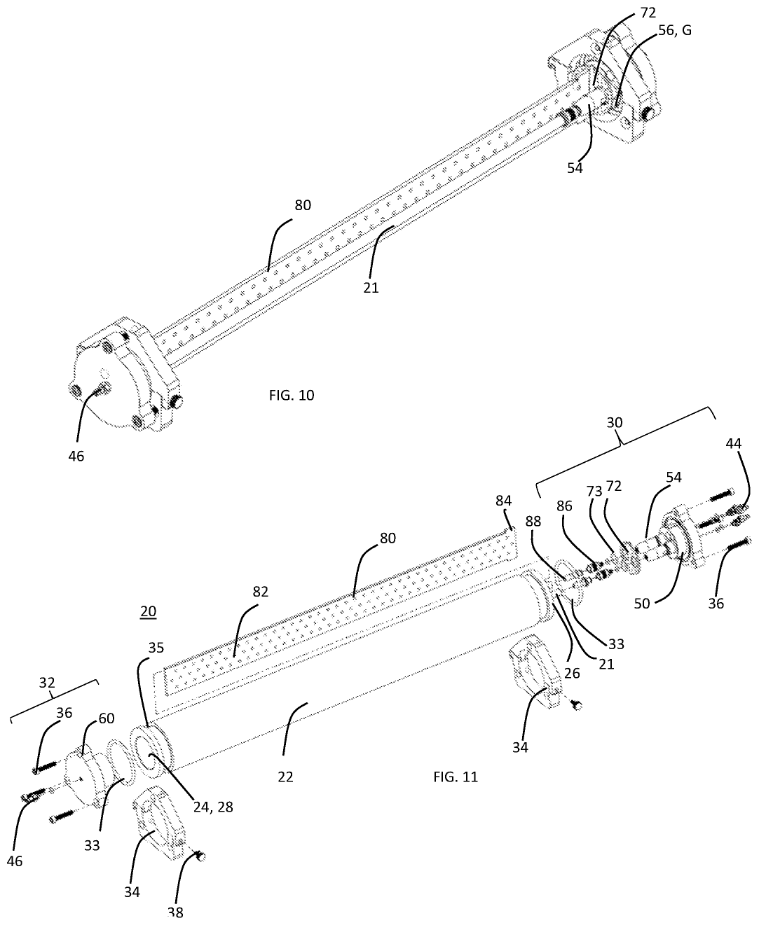

Turning now to FIGS. 10, 11 and 15, system 20 includes a diffuser 72 having a flat and circular body. The body includes two circular openings 74 that are respectively positioned about the protuberances 54 of cap 50. The body of diffuser 72 also includes a series of small openings 76 or perforations that permit the passage of fluid therethrough. Two diametrically opposite tabs 78 extend radially from the outer perimeter of the body.

In an assembled form of system 20, the openings 74 of diffuser 72 are seated against the shoulders 65 of protuberances 54 of cap 50. Seals 73 (FIG. 11) are positioned within the protuberance channels 58 (FIG. 12G) to retain diffuser 72 against shoulders 65. Accordingly, diffuser 72 is held in position, and the gap G is established between diffuser 72 and the ends 59b (FIG. 12F) of passages 52c and 52d. Diffuser 72 divides chamber 28 into two sub-chambers (i.e., a first small sub-chamber at the inlet end, and a second larger sub-chamber consuming the remaining of chamber 28). Also, like the tabs 55 of cap 50, the tabs 78 of diffuser 72 are also positioned within respective slots 64 (FIG. 14) formed at the inlet end 24 of the interior revolved surface of tube 22 such that diffuser 72 is non-rotatably aligned with tube 22. Thus, diffuser 72 is constrained in the radial and axial directions. Along with facilitating alignment, the tabs either limit or prevent fluid from passing around the diffuser and into the tab channels.

Turning now to FIG. 11, system 20 includes a tube separator 80 having a flat and rectangular body that is sized to be positioned within chamber 28 of tube 22. The body of separator 80 also includes a series of small openings 82 or perforations that permit the passage of fluid therethrough. Two tabs 84 extend from the body at the inlet side of separator 80.

In an assembled form of system 20, the tabs 84 of separator 80 are also positioned within respective slots 64 (FIG. 14) formed at the inlet end 24 of the interior revolved surface of tube 22 such that separator 80 is non-rotatably aligned with tube 22. As best shown in FIG. 5, tabs 84 are also sandwiched (or otherwise positioned) in an axial direction between diffuser 72 and an annular shoulder 83 formed on an interior surface of tube 22. Thus, separator 80 is constrained in the radial, circumferential and axial directions.

The components described herein may be formed from any materials known to those skilled in the art (plastic, metal, etc.), and formed using any process known to those skilled in the art (molding, casting, machining, etc.). The materials may be transparent, for example, for permitting an observer to visualize the decellularization process occurring within the system 20.

Turning now to one exemplary method of operating system 20, the system 20 is initially in a partially assembled state whereby inlet cap assembly 30 is pre-assembled and disconnected from tube 22 while outlet cap assembly 32 is connected to tube 22.

The grafts 21 are first connected to respective connectors 88. Specifically, a first graft 21 is connected to the barb connector of one of the connectors 88; and a second graft 21 is connected to the barb connection to the other connector 88. A suture or other device may optionally be used to aid in securing the grafts 21 to the connectors 88. The grafts 21 may (or may not) be laid on the surfaces of separator 80. Separator 80 is then positioned through the inlet end 24 of tube 22 until the tabs 84 of separator 80 are seated within slots 64 (FIG. 14) formed in tube 22. At the same time as the separator 80 is loaded within tube 22, inlet cap assembly 30 is mounted to the inlet end 24 of tube 22. As noted above, to mount inlet cap assembly 30 to tube 22, the tabs 78 (FIG. 15) of diffuser 72 are positioned within respective slots 64 of tube 22. Clamp 34 is then mounted to the inlet end 24 of tube 22; fasteners 36 are fastened to both clamp 34 and inlet cap assembly 30; and clamp fastener 38 is applied to fix the two halves of clamp 34, thereby fixing inlet cap assembly 30 to tube 22.

If not connected already, fluid delivery lines are connected to the inlet fittings 44a-44d. Fitting 44e may or may not be connected to an external line. Outlet fitting 46 may also be connected to a fluid egress line and/or storage vessel. The fluid egress line may be fluidly connected to the fluid delivery lines (by way of a pump for example) for recirculating the fluid back through system 20.

System 20 is then suspended vertically such that inlet cap assembly 30 is positioned at an elevation above outlet cap assembly 32. Grafts 21 are therefore suspended vertically by gravity. System 20 may or may not be enclosed within an incubator for heating the fluid within system 20.

Fluid is then delivered through the fittings 44a-44d. According to one embodiment, the same fluid is delivered through all of the fittings 44a-44d. Specifically, fluid delivered through fittings 44a and 44b is directed through passages 52a and 52b of protuberances 54, respectively, connectors 86 and 88 and into the lumens of grafts 21. The fluid migrates along the length of grafts 21 and also migrates through the walls of the grafts 21. The fluid is then expelled from the free ends 21a (FIG. 9) of the grafts and into the chamber 28 at a location close to or near the outlet end 26 of the tube 22 depending upon the length of the grafts 21.

At the same time, fluid delivered through fittings 44c and 44d is distributed through the enlarged outlets 59b of passages 52c and 52d, respectively, and into the cylindrical space S (FIG. 5; also referred to herein as a small subchamber of chamber 28) between diffuser 72 and surface 56 of cap 50. Fluid accumulates in the space S, and is eventually expelled uniformly through the openings 76 of diffuser 72 and into the chamber 28, whereupon the fluid contacts the exterior surfaces of the grafts 21. Accordingly, the system 20 is configured to deliver the fluid onto every exposed surface of the grafts 21. Although the grafts 21 are at least partially separated by separator 80, fluid in chamber 28 can migrate from one side of the chamber 28 to the other side of the chamber 28 via openings 82 of separator 80.

Fluid in chamber 28 is expelled from system 20 via outlet cap assembly 32 and its outlet fitting 46. As noted above, a line may be connected to outlet fitting 46. Also, the expelled fluid may be recirculated back through the fittings 44a-44d of the inlet cap assembly 30.

It is noted that the foregoing method of operating the system 20 is not necessarily limited to any one of the steps or sequence of steps. Also, the system 20 could be modified to close various fittings 44 in order to process a different number or type of graft (i.e., non-tubular).

By virtue of its components parts, and as verified by computational fluid dynamics software and lab studies, the system 20 achieves a uniform flow and velocity profile along the entire length of the grafts 21. This is due at least in part to the cylindrical space S (FIG. 5), positioning of diffuser 72, openings 76 of diffuser 72, openings 82 of separator 80, and the enlarged outlets 59b of cap 50. Also, system 20 achieves a uniform flow in less space and fluid volume as compared to a gradual flow path expansion.

The diffuser 72 is downstream of the ports 59b to the chamber 28 and provides a partial blockage, causing a recirculation in order to diffuse the incoming jet of fluid from the smaller diameter external tubing being driven by the pump (not shown). It is desirable to have a more uniform velocity proceeding axially down the tube 20 where the grafts 21 are present. This could be accomplished with a long gradual expansion from the tubing inner diameter to the chamber tube inner diameter to avoid flow separation from the wall, but the diffuser 72 accomplishes a similar goal while saving space and reducing fluid volume.

The system 20 is configured to produce a range of pressure within the normal physiological range of the human circulatory system (˜30-180 mmHg) or as desired to achieve the endpoints of the process. However, the system 20 can be engineered to an arbitrarily high pressure capability, if so desired.

It is noted that the flow rates through each fitting 44a-44d may be maintained in a range of 90-120 mL/min, but could vary. According to one aspect, the ratio of solution volume to tissue volume may be greater than 10, for example. And, the operating temperature may be maintained at 0-50 degrees C., for example. To reach higher temperatures (such as 37 degrees C.), the system 20 may be stowed within an incubator.

The system 20 is intended for decellularization of vascular tissue, but could be used to decellularize other tissue types, rinse materials, stain materials, induce chemical reactions in materials, or other purposes that require a consistent flow along the length of the chamber. The system 20 is scalable in size, such that other diameters, lengths, volumes, and possibly shapes may be used. Due to the cost of materials and reagents, power required for heating and pumping fluid, and space constraints, the size and volume of system 20 has been minimized for cost and efficiency. According to one exemplary embodiment, there are four inlet ports and one outlet port, all with an internal diameter of 0.0938 in, with each inlet line being pumped at a speed of 80-115 mL/min (for example) with resulting fluid pressures between 0 and 180 mmHg. The flow rates and size and number of inlet ports may be adjusted, along with the size and number of outlet ports to achieve different pressures and velocities within the chamber. The fluid flow is created in this instance using peristaltic pumps, but can be induced by any pumping mechanism or, alternatively, by gravity. The fluid type, viscosity, and temperature may be any combination that results in a flowable liquid and may have dispersed particles flowing with the fluid.

One goal of tissue decellularization is to provide essentially an immunoprivilaged graft, similar to autologous tissue, through a thorough removal of cells and cell remnants while retaining the composition and structure of the extracellular matrix (ECM). Cryopreserved vascular allografts provide a surgeon with handling characteristics and hemodynamic profile similar to native tissue, however, due to maintenance of cell viability of the donor tissue, there have been instances where they have been shown to have a chronic graft rejection response. An inflammatory response may contribute to a loss of patency due to thrombosis and stenosis from neointimal hyperplasia. In addition, cryopreserved grafts have been shown to elicit a strong humoral immune response in various studies. These grafts can incite the development of anti-human leukocyte antigen class I and II antibodies that remain detectable years after implantation which might complicate future kidney transplantation. By eliminating major histocompatibility antigens, this response may be attenuated. These failure modes can still be present with a localized response due to insufficient decellularization, resulting in graft failure.

Therefore, one objective of the instant invention is to provide homogeneous and sufficient decellularization while maintaining the extracellular matrix and proteins along the entire length of the tube 22. This reduces the potential for an inflammatory and immune response and stenosis associated with cryopreserved and insufficiently decellularized allografts and provide an appropriate environment for repopulation of native cells in the recipient. Additionally, the invention seeks to maintain the appropriate hemodynamic profile throughout the tube 22 to ensure long-term patency in an AV Access graft.

### System Evaluation

The system design was evaluated along the length of the tube 20. The evaluation focused on the following criteria:
(A) Residual DNA (≤10 ng DNA/mg wet tissue)
   i. DNA was measured along the length of the graft.
   ii. Standard deviation of the residual DNA was calculated for each graft processed in either the teardrop design or the current upright design.
   iii. Teardrop average standard deviation of DNA across the length of the graft was 2.0 ng/mg.
   iv. Upright average standard deviation of DNA across the length of the graft was 0.4 ng/mg.
(B) Histological assessment of cells, cell debris.
(C) Histological assessment of matrix components and proteins.
(D) In vivo assessment in a large animal model:
   i. Decellularized grafts were placed as an arteriovenous shunt between the carotid artery and jugular vein of sheep.
   ii. The graft was stuck with an 18-22 gauge needle three times a week to simulate access during hemodialysis treatment.
   iii. 12-weeks post-implantation, histology was conducted at the carotid anastomosis, mid-graft and jugular anastomosis.
   iv. Mild to no thrombin was noted and typically associated with the injury caused by the needle sticks.
   v. No apparent effects noted on biochemical or hematologic parameters:
      i. haptoglobin (systemic inflammation marker for sheep); and
      ii. thrombin markers.
   vi. No major device findings:
      i. 0/6 Thromboembolic
      ii. 0/6 Pseudo/aneurysm
      iii. 0/6 Stenosis
   vii. Mild to no inflammation, typically associated with sutures and not the graft material
   viii. 100% patency
   ix. Biocompatibility and appropriate healing response:

### More Results/Observations:

A. H&E-stained sections revealed the presence of cells repopulating the graft that fully infiltrated the tunica adventitia (TA) and approximately one-half of the tunica media (TM).

B. Cellular infiltrate stained positive for proliferating nuclear cell antigen (PCNA), an intracellular protein that indicates cell replication, and thus, cell viability.

C. A TUNEL assay, which detects DNA fragments occurring during apoptotic cells, or programmed cell death, revealed few positive-stained cells in the graft, further supporting that the cells repopulating the graft were viable.

D. Cells in the graft stained positive for aSMA and HSP47 indicating the cells repopulating the graft expressed proteins that are typical of native cells. Specifically, aSMA is expressed by smooth muscle cells, the predominant cell type of the vascular wall, and HSP47 is a marker for collagens I and III production, the predominant collagen types of the vascular wall.

E. A mild inflammatory infiltrate was also observed, both in the TM and the TA. Altogether, the relatively mild inflammatory response coupled with the effective recellularization, with viable and phenotypically correct cells required for remodeling and healing, demonstrate good biocompatibility of the graft.

F. Movat's staining revealed connective tissue and recipient cells within the TA extending into the TM, and variably into the tunica intima (TI), both at the carotid and jugular anastomoses and in mid-graft sections. Within the TA, organized connective tissue primarily composed of collagen was detected, while in the TM and TI, proteoglycans and αSMA-positive cells were seen. A mild foreign body inflammatory response associated with suture fragments was also noted, which is an expected finding after implantation. The presence of these matrix components and aSMA-positive cells, essential building blocks of the vascular wall, indicate evidence of healing in the grafts.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A system for processing a tubular tissue sample defining a lumen and an exterior surface, said system comprising:
a container having one or more walls defining a hollow interior chamber for receiving the tubular tissue sample;
a first fluid inlet passage associated with the container and configured to direct fluid through the lumen of the tissue sample; and
a second fluid inlet passage associated with the container and configured to direct fluid over the exterior surface of the tissue sample.

2. The system of claim 1, wherein the container is a cylindrical tube.

3. The system of claim 1, further comprising a fluid connector that is either directly or indirectly mounted to the first fluid passage and positioned within the chamber, wherein the fluid connector is sized to fit within the lumen of the tubular tissue sample.

4. The system of claim 1 further comprising a third inlet passage associated with the container and configured to direct fluid through the lumen of a second tissue sample.

5. The system of claim 4 further comprising a perforated separator positioned within the chamber at a location between the first and third inlet passages.

6. The system of claim 1, wherein the second fluid inlet passage extends between a fluid entrance and a fluid exit, wherein a passageway between the fluid entrance and exit has a non-constant cross section and wherein the fluid exit has a larger cross section than the fluid entrance.

7. The system of claim 1, further comprising a first fluid fitting mounted to the first fluid passage for delivering fluid through the first fluid passage, and a second fluid fitting mounted to the second fluid passage for delivering fluid through the second fluid passage.

8. The system of claim 1, wherein an exit port of the second fluid inlet passage is disposed on a surface of the container, and the system further comprises a perforated diffuser that is spaced apart from the surface by a pre-determined distance thereby defining a space between the surface of the container and the diffuser, and wherein the diffuser divides the chamber into a first subchamber in which the space is defined and a second subchamber in which the tissue sample can be positioned.

9. The system of claim 8, wherein the first fluid inlet passage extends into the second subchamber of the chamber while the second fluid inlet passage does not extend into the second subchamber of the chamber.

10. The system of claim 1, further comprising a fluid outlet passage disposed on the container through which the fluid is expelled from the chamber.

11. The system of claim 10, wherein the fluid outlet passage is disposed on a side of the container that is opposite a side of the container on which the first and second fluid inlet passages are disposed.

12. The system of claim 1, further comprising a gas bubble exit passage disposed on a wall of the container.

13. A method for processing a tubular tissue sample, which is stored in a container having one or more walls defining a hollow interior chamber, said method comprising:
delivering fluid through a first fluid inlet passage associated with the container and through a lumen of the tissue sample; and
delivering fluid through a second fluid inlet passage associated with the container and over an exterior surface of the tissue sample.

14. The method of claim 13 further comprising delivering fluid through a fluid connector that is either directly or indirectly mounted to the first fluid passage and positioned within the chamber, wherein the fluid connector is at least partially positioned within the lumen of the tubular tissue sample.

15. The method of claim 13 further comprising delivering fluid through a third inlet passage associated with the container and through the lumen of a second tissue sample.

16. The method of claim 15 further comprising delivering fluid through a perforated separator that is positioned within the chamber at a location between the first and third inlet passages as well as between the first and second tissue samples.

17. The method of claim 13, wherein the second fluid inlet passage extends between a fluid entrance and a fluid exit, and wherein a passageway between the fluid entrance and exit has a non-constant cross section and wherein the fluid exit has a larger cross section than the fluid entrance.

18. The method of claim 13, wherein the second fluid inlet passage has an exit port that is disposed on a surface of the container, and a perforated diffuser is spaced apart from the surface by a pre-determined distance thereby defining a space between the surface of the container and the diffuser, and wherein the diffuser divides the chamber into a first subchamber in which the space is defined and a second subchamber in which the tissue sample is positioned, and the method further comprises distributing the fluid from the first subchamber of the chamber, through the perforated divider and into the second subchamber of the chamber.

19. The method of claim 18, wherein the first fluid inlet passage extends into the second subchamber of the chamber while the second fluid inlet passage does not extend into the second subchamber of the chamber.

20. The method of claim 13, further comprising distributing the fluid out of the chamber via a fluid outlet passage disposed on the container.

21. A tubular tissue product produced by a process comprising:
delivering fluid through a first fluid inlet passage associated with a container and through a lumen of a tissue sample; and
delivering fluid through a second fluid inlet passage associated with the container and over an exterior surface of the tissue sample.

22. A tubular tissue product produced by the method of claim 12.

23. A tubular tissue product produced by the system of claim 1.
